(19) United States
(12) Patent Application Publication
(10) Pub. No.: US 2024/0409532 A1
(43) Pub. Date: Dec. 12, 2024

(54) BIPHENYL SULFONAMIDES AS DUAL ANGIOTENSIN ENDOTHELIN RECEPTOR ANTAGONISTS

(71) Applicants: Nenggang Li, Chongqing (CN); Chongqing Hongyuantang Medicine Co., Ltd., Chongqing (CN)

(72) Inventors: Nenggang Li, Chongqing (CN); Fan Li, Chongqing (CN)

(21) Appl. No.: 18/230,686

(22) Filed: Aug. 7, 2023

(30) Foreign Application Priority Data
Jun. 8, 2023 (CN) 202310676316.1

(51) Int. Cl. C07D 413/14

(52) U.S. Cl. CPC C07D 413/14

(57) ABSTRACT

The present invention provides biphenyl sulfonamide compounds which are combined angiotensin and endothelin receptor antagonists useful in the treatment of conditions such as diabetic nephropathy and other diseases.

### CROSS-REFERENCE TO RELATED APPLICATION

The present application claims priority from Chinese Patent Application No. 202310676316.1 filed on Jun. 8, 2023, the contents of which are incorporated herein by reference in their entirety.

### FIELD OF THE INVENTION

The present invention relates to biphenyl sulfonamide compounds which are combined angiotensin and endothelin receptor antagonists, to methods of using such compounds in the treatment of conditions such as immunoglobulin A nephropathy and other diseases, and to pharmaceutical compositions containing such compounds.

### BACKGROUND OF THE INVENTION

Angiotensin II (AngII) and endothelin-1 (ET-1) are two of the most potent endogenous vasoactive peptides currently known and are believed to play a role in controlling both vascular tone and pathological tissue remodeling associated with a variety of diseases including hypertension, diabetic nephropathy and heart failure. Currently, angiotensin receptor blockers (ARBs), which block the activity of Ang II, are widely used as a treatment for hypertension, diabetic nephropathy and heart failure. In addition, there is a growing body of data that demonstrates the potential therapeutic benefits of ET receptor antagonists (ERAs) in blocking ET-1 activity.

It is also known that Ang II and ET-1 work together in blood pressure control and pathological tissue remodeling. For example, ARBs not only block the action of Ang II at its receptor, but also limit the production of ET-1. Similarly, ERAs block ET-1 activity and inhibit the production of Ang II. Consequently, simultaneously blocking Ang II and ET-1 activities may offer better efficacy than blocking either substance alone.

### SUMMARY OF THE INVENTION

Disclosed herein are compounds of general formula I, and pharmaceutically acceptable salts thereof, that are potent and selective antagonists of both angiotensin and endothelin receptors:

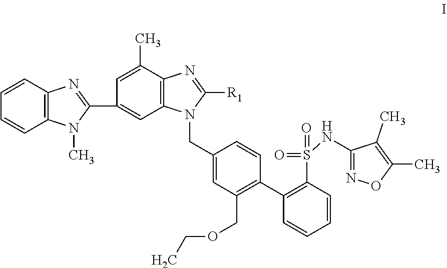

wherein: R1 is hydrogen, (cycloalkyl) alkyl, haloalkyl, alkoxy.

Disclosed herein are compounds of general formula II, and pharmaceutically acceptable salts thereof, that are potent and selective antagonists of both angiotensin and endothelin receptors:

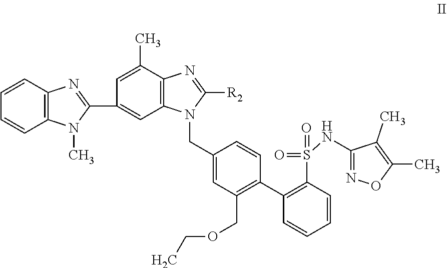

wherein: R2 is hydrogen, (cycloalkyl) alkyl, haloalkyl, alkoxy.

In general, the compounds of the present invention maybe prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. The starting materials, for example in the case of suitably substituted benzimidazole ring compounds, are either commercially available. The starting material of substituted benzimidazole ring compounds, for example “1-1”, “1-5”, “1-8” and “1-11”, can also be prepared using the literature procedures in Journal of Medicinal Chemistry, 1993, 36 (25): 4040-4051. The compounds of “4′-bromomethyl . . . 1,1′-biphenyl . . . ”, for example “1-2” and “2-1”, can also be prepared using the literature procedures in Journal of Medicinal Chemistry, 2005, 48 (1): 171-179.

The biological activity of compounds was evaluated in isolated rat aortic ring and in systemic arterial pressure (SAP) of anesthetized rat experiments using the literature procedures in Chinese Pharmacological Bulletin 2008, 24 (11): 1422-1426.

The compounds of formula I and formula II are antagonists of both endothelin (especially, ET-1) and angiotensin II (especially, subtype AT1) receptors (“dual angiotensin endothelin receptor antagonists”) and are useful in treatment of conditions associated with increased ET levels and/or increased angiotensin II levels and of all endothelin-dependent or angiotensin II-dependent disorders. They are thus useful in treatment of immunoglobulin A nephropathy and other diseases.

The effective amount of a compound of the present invention may be determined by one of ordinary skill in the art, and includes exemplary dosage amounts for a human of from about 0.1 to about 100 mg/kg, preferably about 0.2 to about 50 mg/kg and more preferably from about 0.5 to about 25 mg/kg of body weight (or from about 1 to about 2500 mg, preferably from about 5 to about 500 mg) of active compound per day, which may be administered in a single dose or in the form of individual divided doses, such as from 1 to 4 times per day. It will be understood that the specific dose level and frequency of dosage for any particular subject may be varied and will depend upon a variety of factors including the activity of the specific compound employed, the metabolic stability and length of action of that compound, the species, age, body weight, general health, sex and diet of the subject, the mode and time of administration, rate of excretion, drug combination, and severity of the particular condition. Preferred subjects for treatment include animals, most preferably mammalian species such as humans, and domestic animals such as dogs, cats and the like, subject to endothelin-dependent or angiotensin II-dependent disorders.

### DETAILED DESCRIPTION OF THE PREFERRED EMBODIMENTS

The present invention will be further explained in conjunction with embodiments below, but this does not limit the present invention.

### Example 1

### Synthesis of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-4)

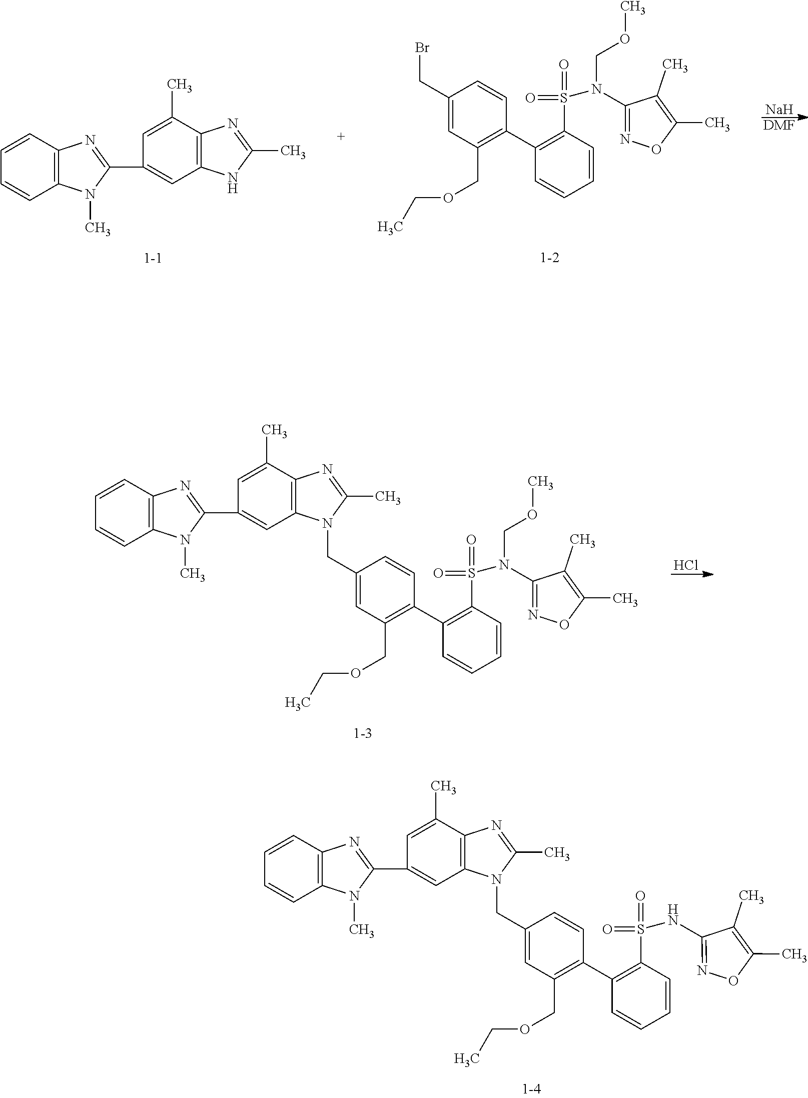

### (i) Synthesis of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-3)

To a solution of 11.0 g (40 mmol) of 1,2′,7′-trimethyl-1H,3′H-2,5′-bibenzo[d]imidazole (1-1) in 60 mL of anhydrous DMF was added 3.2 g of sodium hydride and the mixture stirred at −5° C.˜0° C. for 30 min. A solution of 18.8 g (35.9 mmol) of 4′-(bromomethyl)-N-(4,5-dimethylisoxazol-3-yl)-2′-(ethoxymethyl)-N-(methoxymethyl)-[1,1′-biphenyl]-2-sulfonamide (1-2) in 75 mL of DMF was added and the mixture was stirred at room temperature for a further 1 h. The mixture was diluted with 350 ml of water and the mixture extracted with 3×150 mL of EtOAc. The combined organic extracts were dried (MgSO4) and the solvent was removed in vacuo. The residue was purified by chromatography (50% EtOAc/hexanes) to provide 21.2 g of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-3).

1H NMR (500 MHz, CDCl₃): δ=1.08 (s, 3H), 1.92 (s, 3H), 2.23 (s, 3H), 2.50 (s, 3H), 2.62 (s, 3H), 3.31 (s, 3H), 3.47 (m, 2H), 3.94 (s, 3H), 4.56 (s/m, 2H), 4.60-4.84 (m, 4H), 7.26-8.01 (m, 13H).

MS: m/e=719 (ESI+ mode)

Yield: 21.2 g (82.2%)

### (ii) Synthesis of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-4)

To a 90 mL solution of 5.4 g of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-3) in 95% EtOH was added 75 mL of 6M HCL. The resulting reaction mixture was heated at 100° C. for 2 h. The volatiles were removed in vacuo and the resulting residue was purified by chromatography (25% EtOAc/hexanes) to afford 4.4 g of N-(4,5-dimethylisoxazol-3-yl)-2′-(ethoxymethyl)-N-(methoxymethyl)-4′-((1,2′,7′-trimethyl-1H,3′H-[2,5′-bibenzo[d]imidazol]-3′-yl)methyl)-[1,1′-biphenyl]-2-sulfonamide (1-4).

1H NMR (500 MHz, CDCl₃): δ=1.07 (s, 3H), 1.93 (s, 3H), 2.22 (s, 3H), 2.52 (s, 3H), 2.62 (s, 3H), 3.47 (m, 2H), 3.94 (s, 3H), 4.60-4.84 (m, 4H), 7.24-8.02 (m, 13H).

MS: m/e=675 (ESI+ mode)

Yield: 4.4 g (87.0%)

Molecular formula: C38H38N6O4S

Elemental analysis Found: C, 67.55; H, 5.76; N, 12.51. Calculated: C, 67.63; H, 5.68; N, 12.45.

### Example 2

### Synthesis of N-(4, 5-dimethylisoxazol-3-yl)-2′-(ethoxymethyl)-4′-((1, 2′, 7′-trimethyl-1H,3′H-[2,5′-bibenzo[d]imidazol]-3′-yl)methyl)-[1,1′-biphenyl]-2-sulfonamide (1-7)

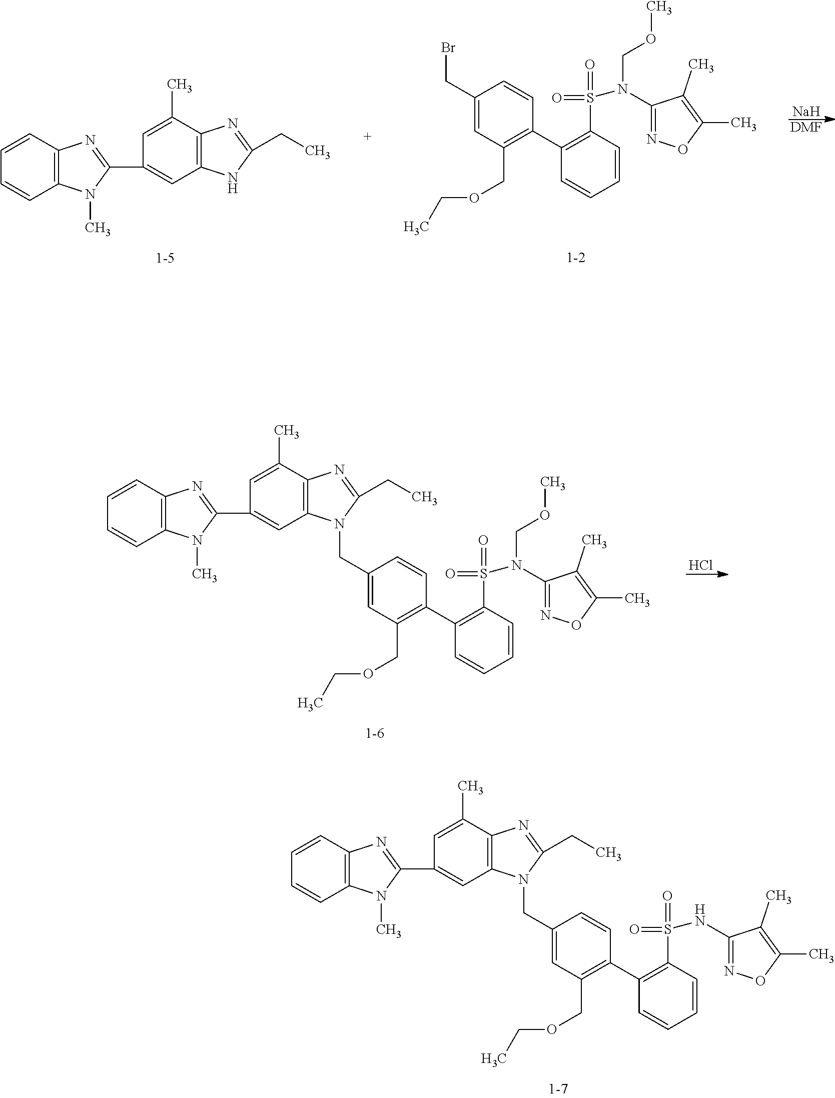

### (i) Synthesis of N-(4,5-dimethylisoxazol-3-yl)-2′-(ethoxymethyl)-4′-((2′-ethyl-1,7′-dimethyl-1H,3′H-[2,5′-bibenzo[d]imidazol]-3′-yl)methyl)-N-(methoxymethyl)-[1,1′-biphenyl]-2-sulfonamide (1-6)

To a solution of 11.6 g (40 mmol) of 2′-ethyl-1, 7′-dimethyl-1H, 3′H-2, 5′-bibenzo[d]imidazole (1-5) in 60 mL of anhydrous DMF was added 3.2 g of sodium hydride (60%, 80 mmol) and the mixture stirred at −5° C.˜0° C. for 30 min. A solution of 18.8 g (35.9 mmol) of 4′-(bromomethyl)-N-(4,5-dimethylisoxazol-3-yl)-2′-(ethoxymethyl)-N-(methoxymethyl)-[1,1′-biphenyl]-2-sulfonamide (1-2) in 75 mL of DMF was added and the mixture was stirred at room temperature for a further 1 h. The mixture was diluted with 350 mL of water and the mixture extracted with 3×150 ml of EtOAc. The combined organic extracts were dried (MgSO4) and the solvent was removed in vacuo. The residue was purified by chromatography (50% EtOAc/hexanes) to provide 22.6 g of N-(4,5-dimethylisoxazol-3-yl)-2′-(ethoxymethyl)-4′-((2′-ethyl-1,7′-dimethyl-1H,3′H-[2,5′-bibenzo[d]imidazol]-3′-yl)methyl)-N-(methoxymethyl)-[1,1′-biphenyl]-2-sulfonamide (1-6).

1H NMR (500 MHz, CDCl₃): δ=1.06 (s, 3H), 1.27 (t, 3H), 1.92 (s, 3H), 2.23 (s, 3H), 2.60 (s, 3H), 2.78 (m, 2H), 3.31 (s, 3H), 3.46 (m, 2H), 3.92 (s, 3H), 4.56 (s, 2H), 4.60-4.84 (m, 4H), 7.26-8.00 (m, 13H)

MS: m/e=733 (ESI+ mode)

Yield: 22.6 g (85.9%)

### (ii) Synthesis of N-(4,5-dimethylisoxazol-3-yl)-2′-(ethoxymethyl)-4′-((2′-ethyl-1,7′-dimethyl-1H,3′H-[2,5′-bibenzo[d]imidazol]-3′-yl)methyl)-[1,1′-biphenyl]-2-sulfonamide (1-7)

To a 90 mL solution of 5.5 g of N-(4,5-dimethylisoxazol-3-yl)-2′-(ethoxymethyl)-4′-((2′-ethyl-1,7′-dimethyl-1H,3′H-[2,5′-bibenzo[d]imidazol]-3′-yl)methyl)-N-(methoxymethyl)-[1,1′-biphenyl]-2-sulfonamide (1-6) in 95% EtOH was added 75 mL of 6M HCl. The resulting reaction mixture was heated at 100° C. for 2 h. The volatiles were removed in vacuo and the resulting residue was purified by chromatography (25% EtOAc/hexanes) to afford 4.2 g of N-(4,5-dimethylisoxazol-3-yl)-2′-(ethoxymethyl)-4′-((2′-ethyl-1,7′-dimethyl-1H,3′H-[2,5′-bibenzo[d]imidazol]-3′-yl)methyl)-[1,1′-biphenyl]-2-sulfonamide (1-7).

1H NMR (500 MHz, CDCl₃): δ=1.07 (s, 3H), 1.25 (t, 3H), 1.92 (s, 3H), 2.23 (s, 3H), 2.62 (s, 3H), 2.78 (m, 2H), 3.47 (m, 2H), 3.94 (s, 3H), 4.60-4.84 (m, 4H), 7.26-8.01 (m, 13H).

MS: m/e=689 (ESI+ mode)

Molecular formula: C39H40N6O4S

Yield: 4.2 g (81.3%)

Elemental analysis Found: C, 68.11; H, 5.86; N, 12.31. Calculated: C, 68.00; H, 5.85; N, 12.20.

### Example 3

### Synthesis of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-10)

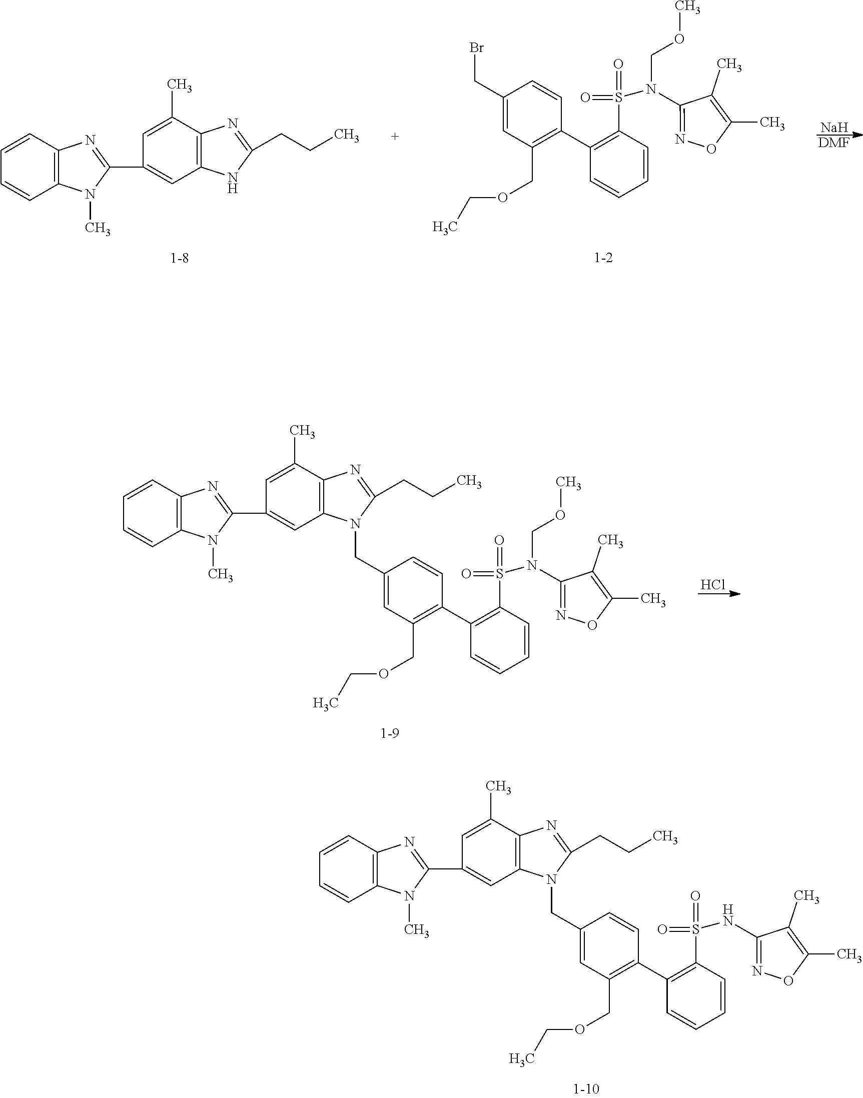

### (i) Synthesis of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-9)

To a solution of 12.2 g (40 mmol) of 1,7′-dimethyl-2′-propyl-1H,3′H-2,5′-bibenzo[d]imidazole (1-8) in 60 mL of anhydrous DMF was added 3.2 g of sodium hydride (60%, 80 mmol) and the mixture stirred at −5° C.˜0° C. for 30 min. A solution of 18.8 g (35.9 mmol) of 4′-(bromomethyl)-N-(4,5-dimethylisoxazol-3-yl)-2′-(ethoxymethyl)-N-(methoxymethyl)-[1,1′-biphenyl]-2-sulfonamide (1-2) in 75 mL of DMF was added and the mixture was stirred at room temperature for a further 1 h. The mixture was diluted with 350 ml of water and the mixture extracted with 3×150 ml of EtOAc. The combined organic extracts were dried (MgSO4) and the solvent was removed in vacuo. The residue was purified by chromatography (50% EtOAc/hexanes) to provide 22.5 g of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-9).

1H NMR (500 MHz, CDCl₃): δ=0.91 (t, 3H), 1.07 (s, 3H), 1.68 (m, 2H), 1.93 (s, 3H), 2.24 (s, 3H), 2.63 (s, 3H), 2.84 (t, 2H), 3.31 (s, 3H), 3.47 (m, 2H), 3.93 (s, 3H), 4.56 (s, 2H), 4.60-4.84 (m, 4H), 7.29-8.04 (m, 13H).

MS: m/e=747 (ESI+ mode)

Yield: 22.5 g (83.9%)

### (ii) Synthesis of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-10)

To a 90 mL solution of 5.6 g of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-9) in 95% EtOH was added 75 mL of 6M HCl. The resulting reaction mixture was heated at 100° C. for 2 h. The volatiles were removed in vacuo and the resulting residue was purified by chromatography (20% EtOAc/hexanes) to afford 4.0 g of 4′-((1,7′-dimethyl-2′-propyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-10).

1H NMR (500 MHz, CDCl₃): δ=0.89 (t, 3H), 1.06 (s, 3H), 1.67 (m, 2H), 1.92 (s, 3H), 2.23 (s, 3H), 2.60 (s, 3H), 2.83 (t, 2H), 3.47 (m, 2H), 3.94 (s, 3H), 4.60-4.84 (m, 4H), 7.26-8.01 (m, 13H).

MS: m/e=703 (ESI+ mode)

Molecular formula: C40H42N6O4S

Yield: 4.0 g (75.9%)

Elemental analysis Found: C, 68.13; H, 5.97; N, 11.85. Calculated: C, 68.35; H, 6.02; N, 11.96.

### Example 4

### Synthesis of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-13)

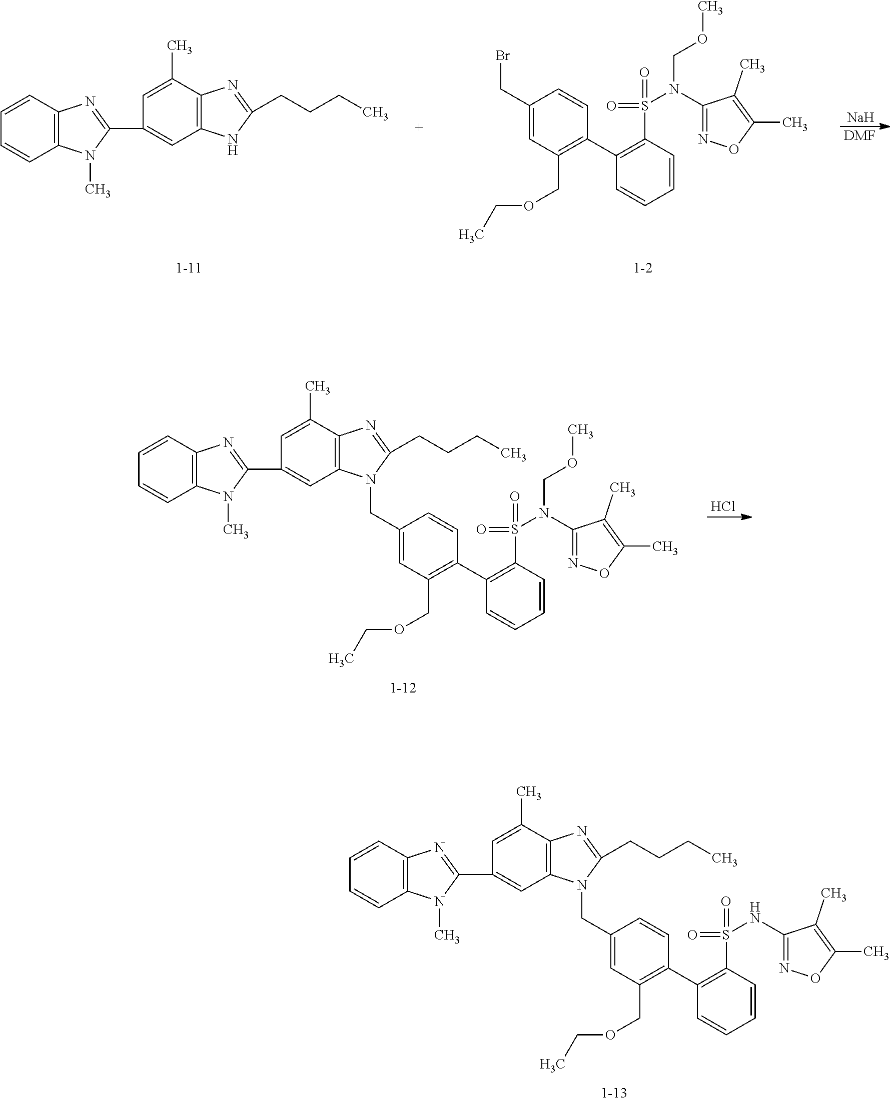

### (i) Synthesis of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-12)

To a solution of 12.7 g (40 mmol) of 2′-butyl-1,7′-dimethyl-1H,3′H-2,5′-bibenzo[d]imidazole (1-11) in 60 mL of anhydrous DMF was added 3.2 g of sodium hydride (60%, 80 mmol) and the mixture stirred at −5° C.˜0° C. for 30 min. A solution of 18.8 g (35.9 mmol) of 4′-(bromomethyl)-N-(4,5-dimethylisoxazol-3-yl)-2′-(ethoxymethyl)-N-(methoxymethyl)-[1,1′-biphenyl]-2-sulfonamide (1-2) in 75 mL of DMF was added and the mixture was stirred at room temperature for a further 1 h. The mixture was diluted with 350 mL of water and the mixture extracted with 3×150 ml of EtOAc. The combined organic extracts were dried (MgSO4) and the solvent was removed in vacuo. The residue was purified by chromatography (50% EtOAc/hexanes) to provide 23.2 g of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-12).

1H NMR (500 MHz, CDCl₃): δ=0.90 (t, 3H), 1.37 (m, 2H), 1.60 (m, 2H), 2.82 (t, 2H), 2.62 (s, 3H), 3.94 (s, 3H), 7.30-7.52 (m, 4H), 7.80 (d, 2H), 4.60-4.84 (m, 4H), 1.07 (s, 3H), 3.47 (m, 2H), 7.26-8.01 (m, 7H), 3.31 (s, 3H), 4.56 (m, 2H), 1.92 (s, 3H), 2.23 (s, 3H),

MS: m/e=761 (ESI+ mode) Yield: 23.2 g (84.9%)
(ii) Synthesis of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-13)

To a 90 mL solution of 5.7 g of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-N-(methoxymethyl)-[1,1′-biphenyl]-2-sulfonamide (1-12) in 95% EtOH was added 75 mL of 6M HCl. The resulting reaction mixture was heated at 100° C. for 2 h. The volatiles were removed in vacuo and the resulting residue was purified by chromatography (20% EtOAc/hexanes) to afford 4.2 g of 4′-((2′-butyl-1,7′-dimethyl-1H,3′H-[2,5′-bibenzo[d]imidazol]-3′-yl)methyl)-N-(4,5-dimethylisoxazol-3-yl)-2′-(ethoxymethyl)-[1,1′-biphenyl]-2-sulfonamide (1-13).

1H NMR (500 MHz, CDCl₃): δ=0.91 (t, 3H), 1.07 (s, 3H), 1.36 (m, 2H), 1.62 (m, 2H), 1.92 (s, 3H), 2.23 (s, 3H), 2.64 (s, 3H), 2.82 (t, 2H), 3.47 (m, 2H), 3.94 (s, 3H), 4.60-4.84 (m, 4H), 7.25-8.02 (m, 13H).

MS: m/e=717 (ESI+ mode)

Yield: 4.2 g (78.1%)

Molecular formula: C41H44N6O4S

Elemental analysis Found C, 68.81; H, 6.09; N, 11.64. Calculated: C, 68.69; H, 6.19; N, 11.72.

### Example 5

### Synthesis of 4′-((1,7′-dimethyl-2′-propyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-3)

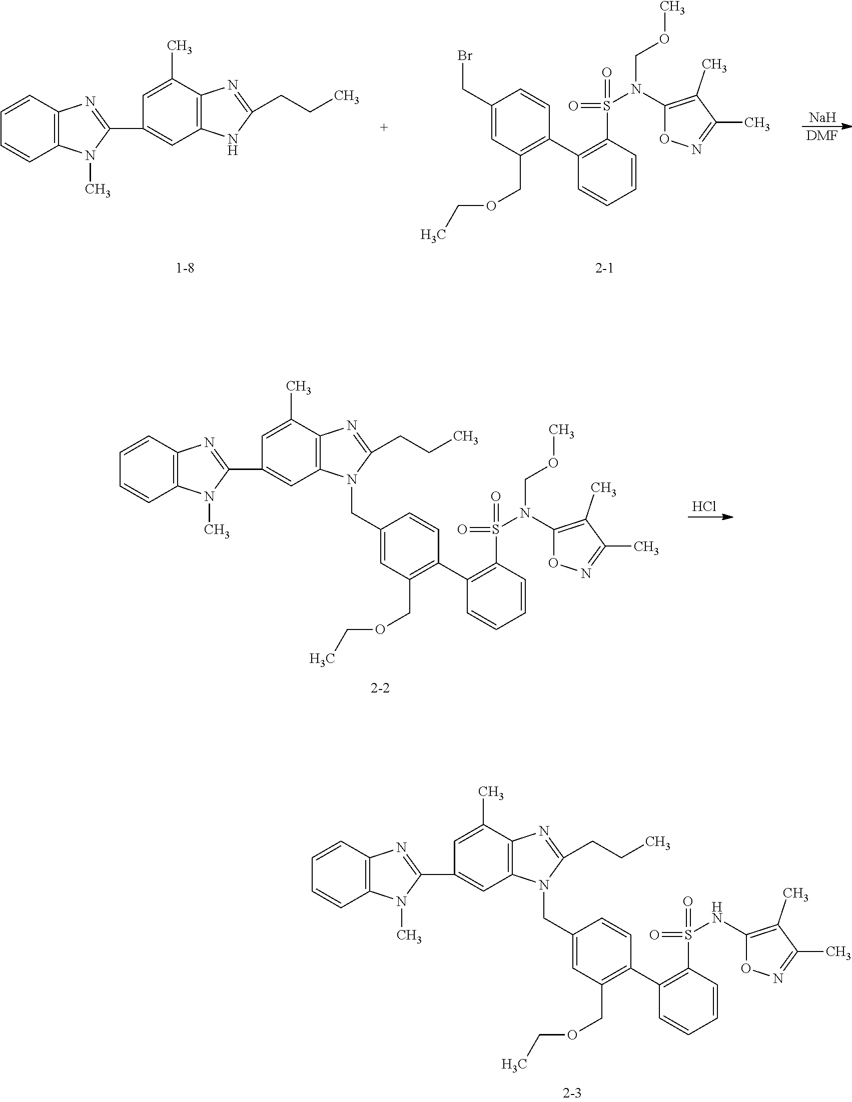

### (i) Synthesis of 4′-((1,7′-dimethyl-2′-propyl-1H,3′H-[2,5′-bibenzo[d]imidazol]-3′-[1,1′-biphenyl]-2-sulfonamide (2-2)

To a solution of 12.2 g (40 mmol) of 1,7′-dimethyl-2′-propyl-1H,3′H-2,5′-bibenzo[d]imidazole (1-8) in 60 mL of anhydrous DMF was added 3.2 g of sodium hydride (60%, 80 mmol) and the mixture stirred at −5° C.˜0° C. for 30 min. A solution of 18.8 g (35.9 mmol) of 4′-(bromomethyl)-N-(3,4-dimethylisoxazol-5-yl)-2′-(ethoxymethyl)-N-(methoxymethyl)-[1,1′-biphenyl]-2-sulfonamide (2-1) in 75 mL of DMF was added and the mixture was stirred at room temperature for a further 1 h. The mixture was diluted with 350 ml of water and the mixture extracted with 3×150 ml of EtOAc. The combined organic extracts were dried (MgSO4) and the solvent was removed in vacuo. The residue was purified by chromatography (50% EtOAc/hexanes) to provide 23.2 g of 4′-((1,7′-dimethyl-2′-propyl-1H,3′H-[2,5′-bibenzo[d]imidazol]-3′-yl)methyl)-N-(3,4-dimethylisoxazol-5-yl)-2′-(ethoxymethyl)-N-(methoxymethyl)-[1,1′-biphenyl]-2-sulfonamide (2-2).

1H NMR (500 MHz, CDCl₃): δ=0.90 (t, 3H), 1.05 (s, 3H), 1.67 (m, 2H), 1.88 (s, 3H), 2.15 (s, 3H), 2.62 (s, 3H), 2.83 (t, 2H), 3.33 (s, 3H), 3.47 (m, 2H), 3.94 (s, 3H), 4.56 (s, 2H), 4.60-4.85 (m, 4H), 7.30-7.52 (m, 4H), 7.80 (d, 2H), 7.26-8.05 (m, 13H)

MS: m/e=747 (ESI+ mode)

Yield: 23.2 g (86.6%)

### (ii) Synthesis of 4′-((1,7′-dimethyl-2′-propyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-3)

To a 90 mL solution of 5.6 g of 4′-((1,7′-dimethyl-2′-propyl-1H,3′H-[2,5′-bibenzo[d]imidazol]-3′-yl)methyl)-N-(3,4-dimethylisoxazol-5-yl)-2′-(ethoxymethyl)-N-(methoxymethyl)-[1,1′-biphenyl]-2-sulfonamide (2-2) in 95% EtOH was added 75 mL of 6M HCl. The resulting reaction mixture was heated at 100° C. for 2 h. The volatiles were removed in vacuo and the resulting residue was purified by chromatography (20% EtOAc/hexanes) to afford 4.2 g of 4′-((1,7′-dimethyl-2′-propyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-3).

1H NMR (500 MHz, CDCl₃): δ=0.92 (t, 3H), 1.07 (s, 3H), 1.63 (m, 2H), 1.88 (s, 3H), 2.13 (s, 3H), 2.62 (s, 3H), 2.83 (t, 2H), 3.47 (m, 2H), 3.92 (s, 3H), 4.60-4.84 (m, 4H), 7.26-8.05 (m, 13H).

MS: m/e=703 (ESI+ mode)

Molecular formula: C40H42N6O4S

Yield: 4.2 g (79.7%)

Elemental analysis Found: C, 68.38; H, 6.08; N, 12.08; Calculated: C, 68.35; H, 6.02; N, 11.96.

### Example 6

### Synthesis of 4′-((2′-butyl-1,7′-dimethyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-5)

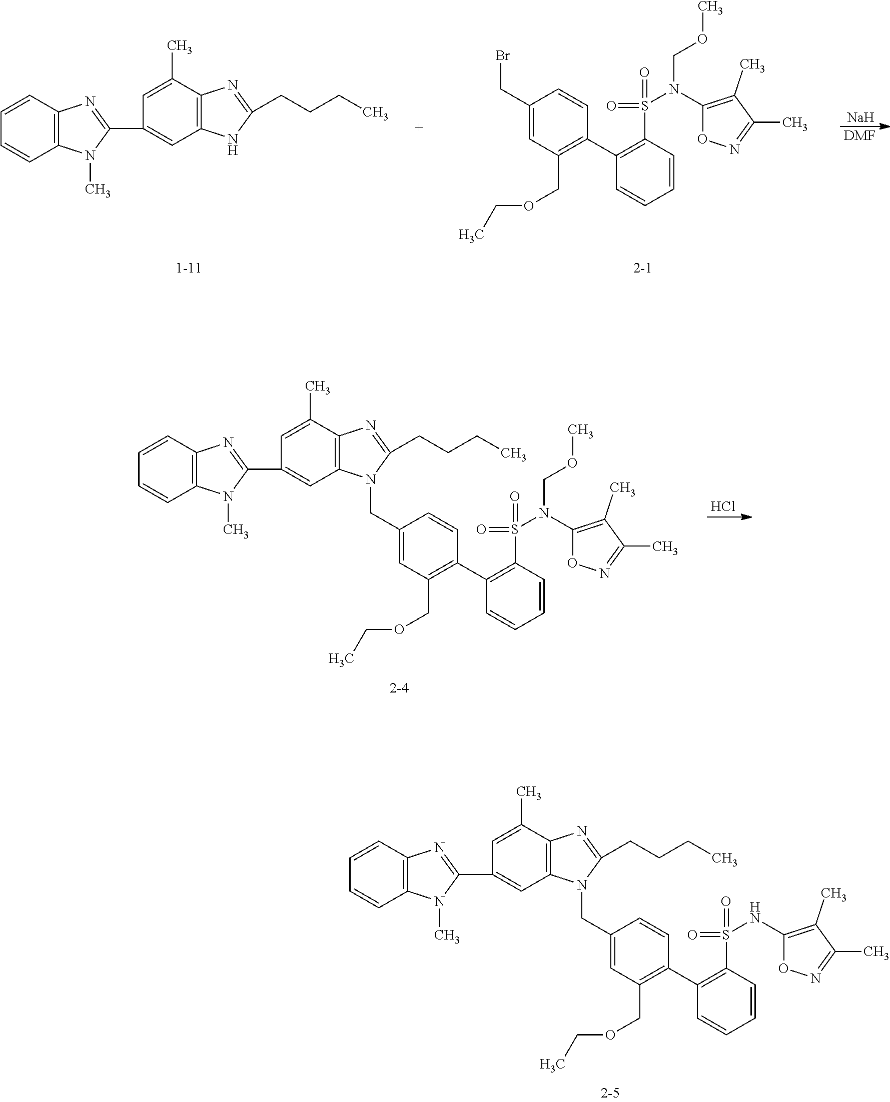

### (i) Synthesis of 4′-((2′-butyl-1,7′-dimethyl-1H,3′H-[2,5′-bibenzo[d]imidazol]-3′-[1,1′-biphenyl]-2-sulfonamide (2-4)

To a solution of 12.7 g (40 mmol) of 2′-butyl-1,7′-dimethyl-1H,3′H-2,5′-bibenzo[d]imidazole (1-11) in 60 mL of anhydrous DMF was added 3.2 g of sodium hydride (60%, 80 mmol) and the mixture stirred at −5° C.˜0° C. for 30 min. A solution of 18.8 g (35.9 mmol) of 4′-(bromomethyl)-N-(3,4-dimethylisoxazol-5-yl)-2′-(ethoxymethyl)-N-(methoxymethyl)-[1,1′-biphenyl]-2-sulfonamide (2-1) in 75 mL of DMF was added and the mixture was stirred at room temperature for a further 1 h. The mixture was diluted with 350 ml of water and the mixture extracted with 3×150 ml of EtOAc. The combined organic extracts were dried (MgSO4) and the solvent was removed in vacuo. The residue was purified by chromatography (50% EtOAc/hexanes) to provide 21.9 g of 4′-((2′-butyl-1,7′-dimethyl-1H,3′H-[2,5′-bibenzo[d]imidazol]-3′-yl)methyl)-N-(3,4-dimethylisoxazol-5-yl)-2′-(ethoxymethyl)-N-(methoxymethyl)-[1,1′-biphenyl]-2-sulfonamide (2-4).

1H NMR (500 MHz, CDCl₃): δ=0.91 (t, 3H), 1.07 (s, 3H), 1.38 (m, 2H), 1.60 (m, 2H), 1.90 (s, 3H), 2.15 (s, 3H), 2.62 (s, 3H), 2.85 (t, 2H), 3.31 (s, 3H), 3.47 (m, 2H), 3.94 (s, 3H), 4.56 (s, 2H), 4.60-4.84 (m, 4H), 7.26-8.01 (m, 13H).

MS: m/e=761 (ESI+ mode)

Molecular formula: C43H48N605S

Yield: 21.9 g (80.2%)

### (ii) Synthesis of 4′-((2′-butyl-1,7′-dimethyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-5)

To a 90 mL solution of 5.7 g of 4′-((2′-butyl-1,7′-dimethyl-1H,3′H-[2,5′-bibenzo[d]imidazol]-3′-yl)methyl)-N-(3,4-dimethylisoxazol-5-yl)-2′-(ethoxymethyl)-N-(methoxymethyl)-[1,1′-biphenyl]-2-sulfonamide (2-4) in 95% EtOH was added 75 mL of 6M HCl. The resulting reaction mixture was heated at 100° C. for 2 h. The volatiles were removed in vacuo and the resulting residue was purified by chromatography (20% EtOAc/hexanes) to afford 4.4 g of 4′-((2′-butyl-1,7′-dimethyl-1H,3′H-[2,5′-bibenzo[d]imidazol]-3′-yl)methyl)-N-(3,4-dimethylisoxazol-5-yl)-2′-(ethoxymethyl)-[1,1′-biphenyl]-2-sulfonamide (2-5).

1H NMR (500 MHz, CDCl₃): δ=0.90 (t, 3H), 1.07 (s, 3H), 1.37 (m, 2H), 1.61 (m, 2H), 1.93 (s, 3H), 2.14 (s, 3H), 2.60 (s, 3H), 2.84 (t, 2H), 3.50 (m, 2H), 3.94 (s, 3H), 4.60-4.84 (m, 4H), 7.28-8.02 (m, 13H).

MS: m/e=717 (ESI+ mode)

Molecular formula: C41H44N6O4S

Yield: 4.4 g (81.8%)

Elemental analysis Found: C, 68.82; H, 6.13; N, 11.87. Calculated: C, 68.69; H, 6.19; N, 11.72.

### In Vitro Evaluation

The biological activity of compounds was evaluated in isolated rat aortic ring and in systemic arterial pressure (SAP) of anesthetized rat experiments using the literature procedures in Chinese Pharmacological Bulletin 2008, 24 (11): 1422-1426.

Telmisartan, as an AT1 receptor antagonist approved for marketing by the US FDA in 1998, was used as a control to evaluate the inhibitory activity of AT1 and ETA receptors. The results are shown in Table 1.

TABLE 1
In Vitro Evaluation Results of the biphenyl sulfonamides
and telmisartan against both AT1 and ETA receptors

| Compound | AT1 Ki(nM) | ETA Ki(nM) |
|---|---|---|
| 1-4 | >102 | >102 |
| 1-7 | 8.9 ± 1.5 | 13.2 ± 3.3 |
| 1-10 | 3.4 ± 1.1 | 6.8 ± 1.8 |
| 1-13 | 2.2 ± 0.8 | 7.2 ± 2.1 |
| 2-3 | 2.1 ± 0.6 | 8.8 ± 2.2 |
| 2-5 | 2.4 ± 0.8 | 9.2 ± 2.3 |
| Telmisartan | 9.5 ± 1.5 | >103 |

1. A compound of the following formula I or a salt or solvate thereof: 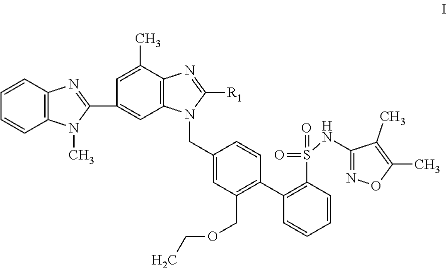
wherein R1 is hydrogen, alkyl, cycloalkyl, or haloalkyl.

2. A compound of the following formula II or a salt or solvate thereof: 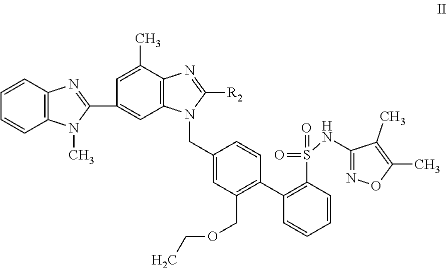
wherein R2 is hydrogen, alkyl, cycloalkyl, or haloalkyl.

3. The compound of claim 1, wherein the compound is selected from the group consisting of the following compounds:

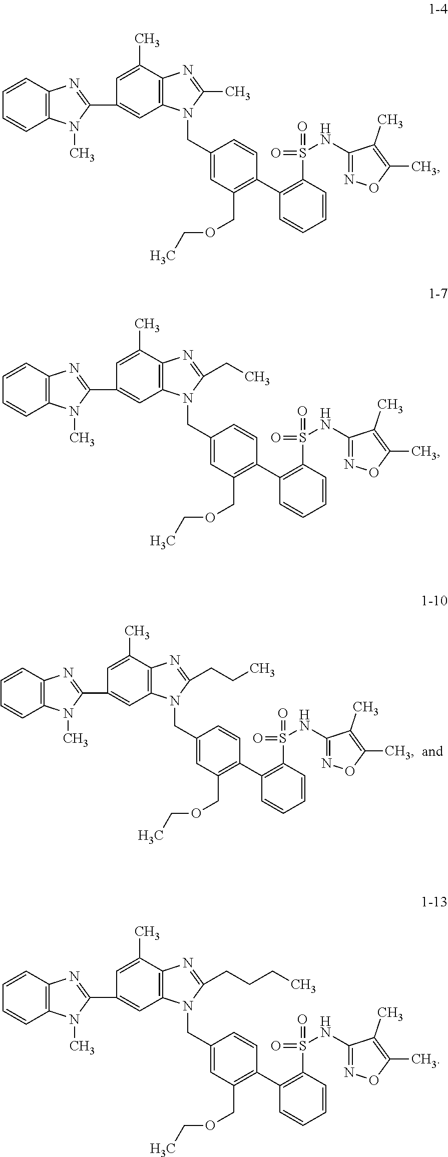 [SMILES: CCOCc1cc(Cn2c(C)nc3c(C)cc(-c4nc5ccccc5n4C)cc32)ccc1-c1ccccc1S(=O)(=O)Nc1noc(C)c1C ; CCOCc1cc(Cn2c(CC)nc3c(C)cc(-c4nc5ccccc5n4C)cc32)ccc1-c1ccccc1S(=O)(=O)Nc1noc(C)c1C ; CCCc1nc2c(C)cc(-c3nc4ccccc4n3C)cc2n1Cc1ccc(-c2ccccc2S(=O)(=O)Nc2noc(C)c2C)c(COCC)c1 ; "and" ; CCCCc1nc2c(C)cc(-c3nc4ccccc4n3C)cc2n1Cc1ccc(-c2ccccc2S(=O)(=O)Nc2noc(C)c2C)c(COCC)c1]

4. The compound of claim 2, wherein the compound is: 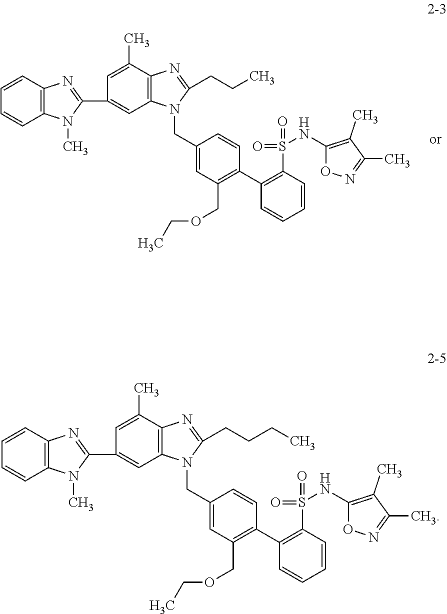 [SMILES: CCCc1nc2c(C)cc(-c3nc4ccccc4n3C)cc2n1Cc1ccc(-c2ccccc2S(=O)(=O)Nc2onc(C)c2C)c(COCC)c1 ; CCCCc1nc2c(C)cc(-c3nc4ccccc4n3C)cc2n1Cc1ccc(-c2ccccc2S(=O)(=O)Nc2onc(C)c2C)c(COCC)c1]
